(19) United States
(12) Patent Application Publication
(10) Pub. No.: US 2004/0014140 A1
(43) Pub. Date: Jan. 22, 2004

(54) Methods for identifying allosteric sites

(76) Inventors: Daniel A. Erlanson, San Francisco, CA (US); Stig K. Hansen, El Cerrito, CA (US); Jeanne Hardy, South San Francisco, CA (US); Joni Lam, Berkeley, CA (US); Thomas O'Brien, San Francisco, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/409,968

(22) Filed: Apr. 8, 2003

Related U.S. Application Data
(60) Provisional application No. 60/370,938, filed on Apr. 8, 2002.

(51) Int. Cl. G01N 33/53

(52) U.S. Cl. 435/7.1

(57) ABSTRACT

The present invention relates to exosites and methods for identifying exosites in protein targets. The present invention also relates to methods for identifying allosteric sites and identifying compounds that bind therein.

### CROSS-REFERENCE TO RELATED APPLICATION

This application claims the benefit of U.S. Provisional Application Serial No. 60/370,938 filed Apr. 8, 2002 which is incorporated herein by reference.

### BACKGROUND

Field of the Invention

Drug discovery targets are often proteins, particularly enzymes. Because most drug discovery efforts rely on mass functional screening to identify lead compounds, potent active site inhibitors are often identified but they rarely become drug candidates. The reasons for the abysmally low success rate are varied but lack of specificity and toxicity play a role more often than not.

An enzymatic target is usually one member of a family of related enzymes. Not surprisingly, related enzymes often share similar three-dimensional structures with each other with the active site region being the most conserved. Because the site being targeted is the region of the enzyme that is most similar among family members, it is not surprising that achieving selectivity against one member is extremely difficult. The lack of specificity often results in toxicity from the inhibition of unintended targets. Despite the inherent problems, many of the most promising drug targets are members of large enzymatic families such as proteases (e.g., aspartyl, cysteine, and serine proteases), kinases and phosphatases. As a result, novel methods for drug discovery are needed against these types of targets that enhance specificity. The present invention provides such methods.

### DESCRIPTION OF THE FIGURES

FIG. 1 is a schematic illustration of one embodiment of the tethering method. A thiol-containing protein is reacted with a plurality of ligand candidates. A ligand candidate that possesses an inherent binding affinity for the target is identified and a ligand is made comprising the identified binding determinant (represented by the circle) that does not include the disulfide moiety.

FIG. 2 is a representative example of two tethering experiments. FIG. 2A is the deconvoluted mass spectrum, of the reaction of thymidylate synthase (“TS”) with a pool of 10 different ligand candidates with little or no binding affinity for TS. FIG. 2B is the deconvoluted mass spectrum of the reaction of TS with a pool of 10 different ligand candidates where one of the ligand candidates possesses an inherent binding affinity to the enzyme.

FIG. 3 is a schematic representation of tethering experiments where the thiol is located at or near two different exosites. FIG. 3A illustrates the situation where the binding of a ligand to the exosite does not affect the function of the target. FIG. 3B illustrates the situation where the binding of a ligand to the exosite does affect the function of the target. In this case, the binding of a ligand to the exosite alters the conformation of the active site such that it inhibits the function of the target. FIG. 3B is an example where the exosite is also an allosteric site.

FIG. 4 is a sequence alignment of selected caspases. The residues that comprise the allosteric site are boxed. The numbers correspond to the numbering in caspase-3.

FIG. 5 is the results of a tethering experiment showing that compound 1 forms a disulfide bond with the small subunit but not the large subunit of caspase-3.

FIG. 6 is the results of an experiment correlating the inhibition of caspase-3 activity with the degree of disulfide formation between caspase-3 and compounds 1 or 2.

FIG. 7 shows unbound (A), substrate-bound (B), and allosterically inhibited (C) forms of caspase-7.

### DESCRIPTION OF THE PREFERRED EMBODIMENTS

The present invention provides methods for identifying novel binding sites on proteins that are referred herein as “exosites” and methods for identifying ligands that bind therein. The ligands themselves identified by the methods herein find use, for example, as lead compounds for the development of novel therapeutic * drugs, enzyme inhibitors, labeling compounds, diagnostic reagents, affinity reagents for protein purification, and the like.

Unless defined otherwise, technical and scientific terms used herein have the same meaning as commonly understood by one of ordinary skill in the art to which this invention belongs. References, such as Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

Definitions

The definition of terms used herein include:

The term “aliphatic” or “unsubstituted aliphatic” refers to a straight, branched, cyclic, or polycyclic hydrocarbon and includes alkyl, alkenyl, alkynyl, cycloalkyl, cycloalkenyl, and cycloalkynyl moieties.

The term “alkyl” or “unsubstituted alkyl” refers to a saturated hydrocarbon.

The term “alkenyl” or “unsubstituted alkenyl” refers to a hydrocarbon with at least one carbon-carbon double bond.

The term “alkynyl” or “unsubstituted alkynyl” refers to a hydrocarbon with at least one carbon-carbon triple bond.

The term “aromatic” or “unsubstituted aromatic” refers to moieties having at least one aryl group. The term also includes aliphatic modified aryls such as alkylaryls and the like.

The term “aryl” or “unsubstituted aryl” refers to mono or polycyclic unsaturated moieties having at least one aromatic ring. The term includes heteroaryls that include one or more heteroatoms within the at least one aromatic ring. Illustrative examples of aryl include: phenyl, naphthyl, tetrahydronaphthyl, indanyl, indenyl, pyridyl, pyrazinyl, pyrimidinyl, pyrrolyl, pyrazolyl, imidazolyl, thiazolyl, oxazolyl, isooxazoly, thiadiazolyl, oxadiazolyl, thiophenyl, furanyl, quinolinyl, isoquinolinyl, and the like.

The term “substituted” when used to modify a moiety refers to a substituted version of the moiety where at least one hydrogen atom is substituted with another group including but not limited to: aliphatic; aryl, alkylaryl, F, Cl, I, Br, —OH; —NO₂; —CN; —CF₃; —CH₂CF₃; —CH₂Cl; —CH₂OH; —CH₂CH₂OH; —CH₂NH₂; —CH₂SO₂CH₃; —OR^{X}; —C(O)R^{X}; —COOR^{X}; —C(O)N(R^{X})₂; —OC(O)R^{X}; —OCOOR^{X}; —OC(O)N(R^{X})₂; —N(R^{X})₂; —S(O)₂R^{X}; and —NR^{X}C(O)R^{X} where each occurrence of Rx is independently hydrogen, substituted aliphatic, unsubstituted aliphatic, substituted aryl, or unsubstituted aryl. Additionally, substitutions at adjacent groups on a moiety can together form a cyclic group.

The term “allosteric site” refers to an exosite wherein the binding of a ligand to this site modulates the activity or function of the protein.

The term “antagonist” is used in the broadest sense and includes any ligand that partially or fully blocks, inhibits or neutralizes a biological activity exhibited by a target, such as a TBM. In a similar manner, the term “agonist” is used in the broadest sense and includes any ligand that mimics a biological activity exhibited by a target, such as a TBM, for example, by specifically changing the function or expression of such TBM, or the efficiency of signaling through such TBM, thereby altering (increasing or inhibiting) an already existing biological activity or triggering a new biological activity.

The term “exosite” is a binding site on a protein that is not its primary binding site. For example, the primary binding site on an enzyme is the active site. The primary binding site on a receptor is the ligand-binding site.

The term “ligand candidate” or “candidate ligand” refers to a compound that possesses or has been modified to possess a reactive group that is capable of forming a covalent bond with a complimentary or compatible reactive group on a target. The reactive group on either the ligand candidate or the target can be masked with, for example, a protecting group.

The term “polynucleotide”, when used in singular or plural, generally refers to any polyribonucleotide or polydeoxribonucleotide, which may be unmodified RNA or DNA or modified RNA or DNA. Thus, for instance, pQlynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions A may be from the same molecule or from different molecules. The regions may * include all of one or more of the molecules, but more typically involve only a region of some of the molecules. One of the molecules of a triple-helical region often is an oligonucleotide. The term “polynucleotide” specifically includes DNAs and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The phrase “protected thiol” or “masked thiol” as used herein refers to a thiol that has been reacted with a group or molecule to form a covalent bond that renders it less reactive and which may be deprotected to regenerate a free thiol.

The phrase “reversible covalent bond” as used herein refers to a covalent bond that can be broken, preferably under conditions that do not denature the target. Examples include, without limitation, disulfides, Schiff-bases, thioesters, coordination complexes, boronate esters, and the like.

The phrase “reactive group” is a chemical group or moiety providing a site at which a covalent bond can be made when presented with a compatible or complementary reactive group. Illustrative examples are —SH that can react with another —SH or —SS— to form respectively a disulfide or a disulfide exchange; an —NH₂ that can react with an activated —COOH to form an amide; an —NH₂ that can react with an aldehyde or ketone to form a Schiff base and the like.

The phrase “reactive nucleophile” as used herein refers to a nucleophile that is capable of forming a covalent bond with a compatible functional group on, another molecule under conditions that do not denature or damage the target. The most relevant nucleophiles are thiols, alcohols, and amines. Similarly, the phrase “reactive electrophile” as used herein refers to an electrophile that is capable of forming a covalent bond with a compatible functional group on another molecule, preferably under conditions that do not denature or otherwise damage the target. The most relevant electrophiles are alkyl halides, imines, carbonyls, epoxides, aziridies, sulfonates, disulfides, activated esters, activated carbonyls, and hemiacetals.

The phrase “site of interest” refers to any site on a target to which a ligand can bind. As used herein, a site of interest is any site that is outside of the primary binding site of a protein. For example, if a target is an enzyme, a site of interest is a site that is not the active site. If a target is a receptor, a site of interest is a site that is not the binding site of the receptor's ligand.

The terms “target,” “Target Molecule,” and “TM” are used interchangeably and in the broadest sense, and refer to a chemical or biological entity for which the binding of a ligand has an effect on the function of the target. The target can be a molecule, a portion of a molecule, or an aggregate of molecules. The binding of a ligand may be reversible or irreversible. Specific examples of target molecules include polypeptides or proteins such as enzymes and receptors, transcription factors, ligands for receptors such growth factors and cytokines, immunoglobulins, nuclear proteins, signal transduction components (e.g., kinases, phosphatases), polynucleotides, carbohydrates, glycoproteins, glycolipids, and other macromolecules, such as nucleic acid-protein complexes, chromatin or ribosomes, lipid bilayer-containing structures, such as membranes, or structures derived from membranes, such as vesicles. The definition specifically includes Target Biological Molecules (“TBMs”) as defined below.

A “Target Biological Molecule” or “TBM” as used herein refers to a single biological molecule or a plurality of biological molecules capable of forming a biologically relevant complex with one another for which a small molecule agonist or antagonist has an effect on the function of the TBM. In a preferred embodiment, the TBM is a protein or a portion thereof or that comprises two or more amino acids, and which possesses or is capable of being modified to possess a reactive group that is capable of forming a covalent bond with a compound having a complementary reactive group. Preferred TBMs include: cell surface and soluble receptors and their ligands; steroid receptors; hormones; immunoglobulins; clotting factors; nuclear proteins; transcription factors; signal transduction molecules; cellular adhesion molecules, co-stimulatory molecules, chemokines, molecules involved in mediating apoptosis, enzymes, and proteins associated with DNA and/or RNA synthesis or degradation.

Many TBMs are those that participate in a receptor-ligand binding interaction and can be either member of a receptor-ligand pair. Illustrative examples of growth factors and their respective receptors include those for: erythropoietin (EPO), thrombopoietin (TPO), angiopoietin (ANG), granulocyte colony stimulating factor (G-CSF), granulocyte macrophage colony stimulating factor (GM-CSF), epidermal growth factor (EGF), heregulin-a and heregulin-b, vascular endothelial growth factor (VEGF), placental growth factor (PLGF), transforming growth factors (TGF-a and TGF-b), nerve growth factor (NGF), neurotrophins, fibroblast growth factor (FGF), platelet-derived growth factor (PDGF), bone morphogenetic protein (BMP), connective tissue growth factor (CTGF), hepatocyte growth factor (HGF), and insulin-like growth factor 1 (IGF-1). Illustrative examples of hormones and their respective receptors include those for: growth hormone, prolactin, placental lactogen (LPL), insulin, follicle stimulating hormone (FSH), luteinizing hormone (LH), and neurokinin-1. Illustrative examples of cytokines and their respective receptors include those for: ciliary neurotrophic factor (CNTF), oncostatin M (OSM), TNF-a; CD40L, stem cell factor (SCF); interleukin-1, interleukin-2, interleukin-4, interleukin-5, interleukin-6, interleukin-8, interleukin-9, interleukin-13, and interleukin-18.

Other TBMs include: cellular adhesion molecules such as CD2, CD11a, LFA-1, LFA-3, ICAM-5, VCAM-1, VCAM-5, and VLA-4; costimulatory molecules such as CD28, CTLA-4, B7-1; B7-2, ICOS, and B7RP-1; chemokines such as RANTES and MIP1b; apoptosis factors such as APAF-1, p53, bax, bak, bad, bid, and c-abl; anti-apoptosis factors such as bcl2, bcl-x(L), and mdm2; transcription modulators such as AP-1 and AP-2; signaling proteins such as TRAF-1, TRAF-2, TRAF-3, TRAF-4, TRAF-5, and TRAF-6; and adaptor proteins such as grb2, cbl, shc, nck, and crk.

Enzymes are another class of preferred TBMs and can be categorized in numerous ways including as: allosteric enzymes; bacterial enzymes (isoleucyl tRNA synthase, peptide deformylase, DNA gyrase, and the like); fungal enzymes (thymidylate synthase and the like); viral enzymes (HIV integrase, HSV protease, Hepatitis C helicase, Hepatitis C protease, rhinovirus protease and the like); kinases (serine/threonine, tyrosine, and dual specificity); phosphatases (serine/threonine, tyrosine, and dual specificity); and proteases (aspartyl, cysteine, metallo, and serine proteases). Notable subclasses of enzymes include: kinases such as Lck, Syk, Zap-70, JAK, FAK, ITK, BTK, MEK, MEKK, GSK-3, Raf, tgf-b-activated kinase-1 (TAK-1), PAK-1, cdk4, Akt, PKC q, IKK b, IKK-2, PDK, ask, nik, MAPKAPK, p9orsk, p70s6k, and P13-K (p85 and p110 subunits); phosphatases such as CD45, LAR, RPTP-a, RPTP-m, Cdc25A, kinase-associated phosphatase, map kinase phosphatase-1, PTP-1B, TC-PTP, PTP-PEST, SHP-1 and SHP-2; caspases such as caspases-1, -3, -7, -8, -9, and -11; and cathespins such as cathepsins B, F, K, L, S, and V. Other enzymatic targets include: BACE, TACE, cytosolic phospholipase A2 (cPLA2), PARP, PDE I-VII, Rac-2, CD26, inosine monophosphate dehydrogenase, 15-lipoxygenase, acetyl CoA carboxylase, adenosylmethionine decarboxylase, dihydroorotate dehydrogenase, leukotriene A4 hydrolase, and nitric oxide synthase.

Exosites

The present invention provides methods for identifying novel binding sites on proteins that are referred herein as “exosites.” These exosites are binding sites on a target protein that are distinct from the primary binding region of the particular target protein. For example, an exosite on an enzyme is any binding site that is not the active site. Similarly, an exosite on a receptor is any binding site that is not a binding site of the receptor's ligand.

In one embodiment, the exosite of interest is an adaptive binding site in a protein. The term “adaptive” is used to refer to these sites because unlike well-defined pockets such as active sites, an adaptive binding site is not apparent in the absence of a ligand. The presence of a ligand induces a conformational change in one or more side chains of the protein to create a binding site in which a ligand ultimately can bind.

In another embodiment, the exosite of interest is an allosteric site. In other words, the binding of a ligand to such a site in a target protein modulates the function of that target protein. The modulation can be both negative as well as positive. For example, when the modulation is negative, the binding of a ligand to an exosite inhibits the function of the target. When the modulation is positive, the binding of a ligand to an exosite enhances (or amplifies) the function of the target. Allosteric sites are often recognition sites for accessory and/or regulatory proteins.

In yet another embodiment, the exosite of interest is both an adaptive binding site and an allosteric site.

The Tethering Method

Tethering is a method of ligand identification that relies upon the formation of a covalent bond between a reactive group on a target and a complimentary reactive group on a potential ligand. The tethering method is described in U.S. Pat. No. 6,335,155; PCT Publication Nos. WO 00/00823 and WO 02/42773; U.S. Ser. No. 10/121,216 entitled METHODS FOR LIGAND DISCOVERY by inventors Daniel Erlanson, Andrew Braisted, and James Wells (corresponding PCT Application No. US02/13061); and Erlanson et al., Proc. Nat. Acad. Sci USA 97:9367-9372 (2000), which are all incorporated by reference. The resulting covalent complex is termed a target-ligand conjugate. Because the covalent bond is formed at a pre-determined site on the target (e.g., a native or non-native cysteine), the stoichiometry and binding location are known for ligands that are identified by this method.

Once formed, the ligand portion of the target-ligand conjugate can be identified using a number of methods. In preferred embodiments, mass spectrometry is used. Mass spectrometry detects molecules based on mass-to-charge ratio (m/z) and can resolve molecules based on their sizes (reviewed in Yates, Trends Genet. 16: 5-8 [2000]). The target-ligand conjugate can be detected directly in the mass spectrometer or fragmented prior to detection. Alternatively, the compound can be liberated within the mass spectrophotometer and subsequently identified. Moreover, mass spectrometry can be used alone or in combination with other means for detection or identifying the compounds covalently bound to the target. Further descriptions of mass spectrometry techniques include Fitzgerald and Siuzdak, Chemistry & Biology 3: 707-715 [1996]; Chu et al., J. Am. Chem. Soc. 118: 7827-7835 [1996]; Siudzak, Proc. Natl. Acad. Sci. USA 91: 11290-11297 [1994]; Burlingame et al., Anal. Chem. 68: 599R-651R [1996]; Wu et al., Chemistry & Biology 4: 653-657 [1997]; and Loo et al., Am. Reports Med. Chem. 31: 319-325 [1996]).

Alternatively, the target-ligand conjugate can be identified using other means. For example, one can employ various chromatographic techniques such as liquid chromatography, thin layer chromatography and the like for separation of the components of the reaction mixture so as to enhance the ability to identify the covalently bound molecule. Such chromatographic techniques can be employed in combination with mass spectrometry or separate from mass spectrometry. One can also couple a labeled probe (fluorescently, radioactively, or otherwise) to the liberated compound so as to facilitate its identification using any of the above techniques. In yet another embodiment, the formation of the new bonds liberates a labeled probe, which can then be monitored. A simple functional assay, such as an ELISA or enzymatic assay can also be used to detect binding when binding occurs in an area essential for what the assay measures. Other techniques that may find use for identifying the organic compound bound to the target molecule include, for example, nuclear magnetic resonance (NMR), surface plasmon resonance (e.g., BIACORE), capillary electrophoresis, X-ray crystallography, and the like, all of which will be well known to those skilled in the art.

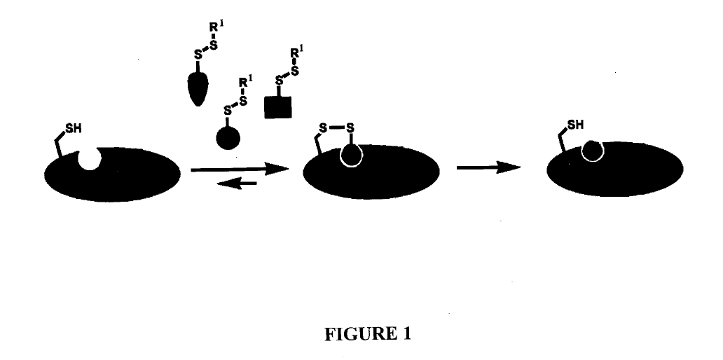

A schematic representation of one embodiment of the tethering method where the target is a protein and the covalent bond is a disulfide is shown in FIG. 1. A thiol containing protein is reacted with a plurality of ligand candidates. In this embodiment, the ligand candidates possess a masked thiol in the form of a disulfide of the formula —SSR¹ where R¹ is unsubstituted C₁-C₁₀ alkyl, substituted C₁-C₁₀ alkyl, unsubstituted aryl or substituted aryl. In certain embodiments, R¹ is selected to enhance the solubility of the potential ligand candidates. As shown, a ligand candidate that possesses an inherent binding affinity for the target is identified and a corresponding ligand that does not include the disulfide moiety is made comprising the identified binding determinant (represented by the circle).

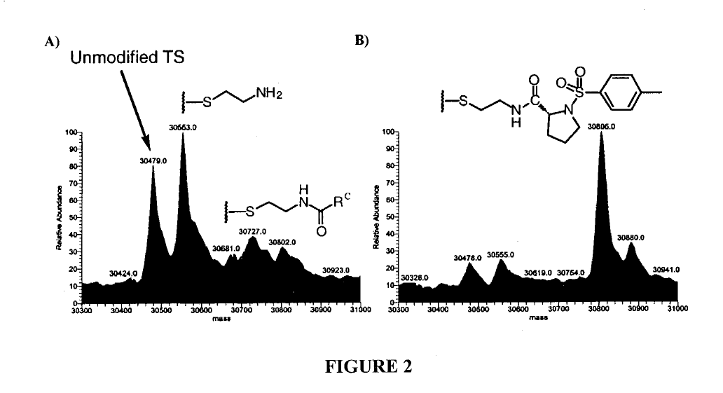

FIG. 2 illustrates two representative tethering experiments where a target enzyme, E. coli thymidylate synthase, is contacted with ligand candidates of the formula

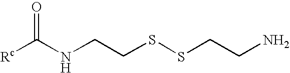

wherein R^{C} is the variable moiety among this pool of library members and is unsubstituted aliphatic, substituted aliphatic, unsubstituted aryl, or substituted aryl. Like all TS enzymes, E. coli TS has an active site cysteine (Cys146) that can be used for tethering. Although the E. coli TS also includes four other cysteines, these cysteines are buried and were found not to be reactive in tethering experiments. For example, in an initial experiment, wild type E. coli TS and the C146S mutant (wherein the cysteine at position 146 has been mutated to serine) were contacted with cystamine, H₂NCH₂CH₂SSCH₂CH₂NH₂. The wild type TS enzyme reacted cleanly with one equivalent of cystamine while the mutant TS did not react indicating that the cystamine was reacting with and was selective for Cys146.

FIG. 2A is the deconvoluted mass spectrum of the reaction of TS with a pool of 10 different ligand candidates with little or no binding affinity for TS. In the absence of any binding interactions, the equilibrium in the disulfide exchange reaction between TS and an individual ligand candidate is to the unmodified enzyme. This is schematically illustrated by the following equation.

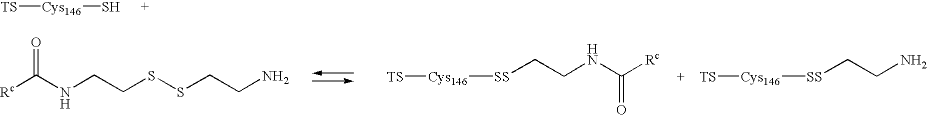

As expected, the peak that corresponds to the unmodified enzyme is one of two most prominent peaks in the spectrum. The other prominent peak is TS where the thiol of Cys146 has been modified with cysteamine. Although this species is not formed to a significant extent for any individual library member, the peak is due to the cumulative effect of the equilibrium reactions for each member of the library pool. When the reaction is run in the presence of a thiol-containing reducing agent such as 2-mercaptoethanol, the active site cysteine can also be modified with the reducing agent. Because cysteamine and 2-mercaptoethanol have similar molecular weights, their respective disulfide bonded TS enzymes are not distinguishable under the conditions used in this experiment. The small peaks on the right correspond to discreet library members. Notably, none of these peaks are very prominent. FIG. 2A is characteristic of a spectrum where none of the ligand candidates possesses an inherent binding affinity for the target.

FIG. 2B is the deconvoluted mass spectrum of the reaction of TS with a pool of 10 different ligand candidates where one of the ligand candidates possesses an inherent binding affinity to the enzyme. As can be seen, the most prominent peak is the one that corresponds to TS where the thiol of Cys146 has been modified with the N-tosyl-D-proline compound. This peak dwarfs all others including those corresponding to the unmodified enzyme and TS where the thiol of Cys146 has been modified with cysteamine. FIG. 2B is an example of a mass spectrum where tethering has captured a moiety that possesses a strong inherent binding affinity for the desired site.

Exosite Identification Using Tethering

In one aspect of the present invention, methods are provided for identifying exosites on protein targets. In general, the method comprises:

a) providing a target comprising a primary binding site and a chemically reactive group at or near a site other than the primary binding site;

b) contacting the target with a compound that is capable of forming a covalent bond with the chemically reactive group;

c) forming a covalent bond between the target and the compound thereby forming a target-compound conjugate; and,

d) determining whether the compound binds to the target at the site in the absence of a covalent bond with the target.

In many cases, potential exosites are located in concave regions in a target. In other cases, potential exosites are not apparent because the sites are adaptive binding sites where the presence of a ligand induces a conformational change in one or more side chains of the protein to create a binding site in which the ligand ultimately can bind.

When the target is an enzyme, the primary binding site is the active site. When the target is a receptor, the primary binding site is the site where the receptor's ligand binds.

A chemically reactive group is considered near a binding site if that group is 10 Angstroms or less from any atom of a residue that comprises that binding site. In another embodiment, the chemically reactive group is considered near a binding site if that group is 5 Angstroms or less from any atom of a residue that comprises that binding site.

In another embodiment, the method comprises:

a) providing a target comprising a first binding site, a second binding site, and a chemically reactive group at or near the second binding site;

b) contacting the target with a first compound that is capable of forming a covalent bond with the chemically reactive group;

c) forming a covalent bond between the target and the first compound thereby forming a target-compound conjugate;

d) contacting the target with a second compound wherein the second compound is a version of the first compound that lacks the chemically reactive group; and,

e) determining the affinity of the second compound for binding non-covalently to the second binding site.

In another aspect of the present invention, methods for identifying an allosteric exosite are provided. In general, the method comprises:

a) providing a target comprising a primary binding site and a chemically reactive group at or near a site other than the primary binding site;

b) contacting the target with a compound that is capable of forming a covalent bond with the chemically, reactive group;

c) forming a covalent bond between the target and the compound thereby forming a target-compound conjugate;

d) determining whether the target-compound conjugate possesses a change in the primary binding site as compared with the target.

The allosteric sites identified by this method can modulate the function of the target protein both negatively as well as positively. For example, when the modulation is negative, the binding of a ligand to an exosite inhibits the function of the target. When the modulation is positive, the binding of a ligand to an exosite enhances (or amplifies) the function of the target.

In one embodiment, the change in the primary binding site is a structural change and is an alteration in the three dimensional structure of the primary binding site. An alteration in the three dimensional structure is defined as a movement of at least one heteroatom of an active site residue by at least 1 Angstrom. The change in structure is detected in any number of ways including x-ray crystallography, NMR, circular dichroism, and the like. In another embodiment, the change in the primary binding site is a functional one. If the target is an enzyme, its function can be either inhibited or enhanced. If the target is a receptor, the binding of the receptor ligand to its binding site can be either inhibited or enhanced.

In another embodiment, the method comprises:

a) providing a target comprising a first binding site, a second binding site, and a chemically reactive group at or near the second binding site;

b) contacting the target with a compound that is capable of forming a covalent bond with the chemically reactive group;

c) forming a covalent bond between the target and the compound thereby forming a target-compound conjugate;

d) determining whether the target-compound conjugate possesses a change in the first binding site as compared with the first binding site of the target.

In certain embodiments of the inventive methods, the chemically reactive group is a thiol of a cysteine residue and the compound possesses a —SH group. In other embodiments, the compound possesses a masked thiol. In other embodiments, the compound is a ligand candidate possessing a masked thiol in the form of a disulfide of the formula —SSR¹ where R¹ is unsubstituted C₁-C₁₀ aliphatic, substituted C₁-C₁₀ aliphatic, unsubstituted aryl or substituted aryl. In other embodiments, the ligand candidate possesses a thiol masked as a disulfide of the formula —SSR²R³ wherein R² is C₁-C₅ alkyl (preferably —CH₂—, —CH₂CH₂—, or —CH₂CH₂CH₂—) and R³ is NH₂, OH, or COOH. Illustrative examples of ligand candidates include:

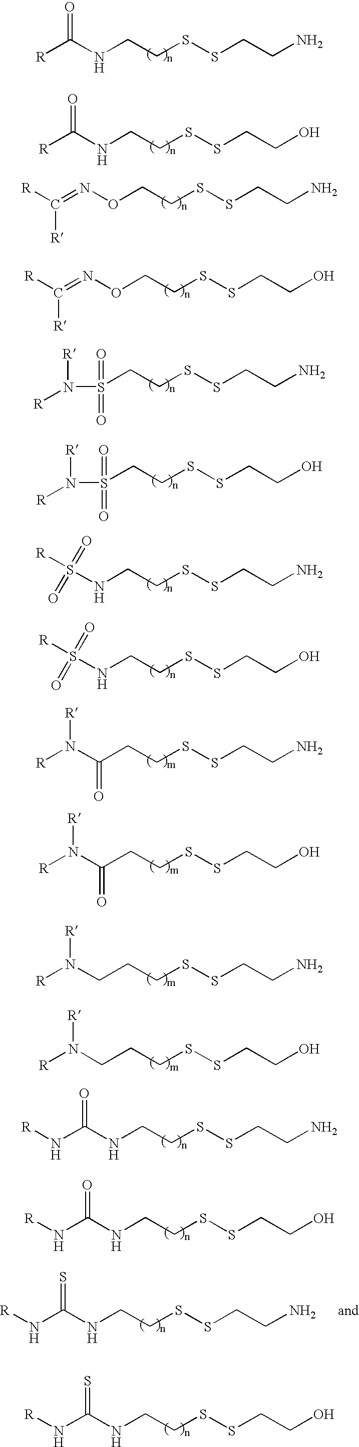

where R and R′ are each independently unsubstituted C₁-C₂₀ aliphatic, substituted C₁-C₂₀ aliphatic, unsubstituted aryl, or substituted aryl; m is 0, 1, or 2; and n is 1 or 2.

In other embodiments, the target is contacted with a compound that is capable of forming a disulfide bond in the presence of a reducing agent. Illustrative examples of suitable reducing agents include but are not limited to: cysteine, cysteamine, dithiothreitol, dithioerythritol, glutathione, 2-mercaptoethanol, 3-mercaptoproprionic acid, a phosphine such as tris-(2-carboxyethyl-phosphine) (“TCEP”), or sodium borohydride. In one embodiment, the reducing agent is 2-mercaptoethanol. In another embodiment, the reducing agent is cysteamine. In another embodiment, the reducing agent is glutathione. In another embodiment, the reducing agent is cysteine.

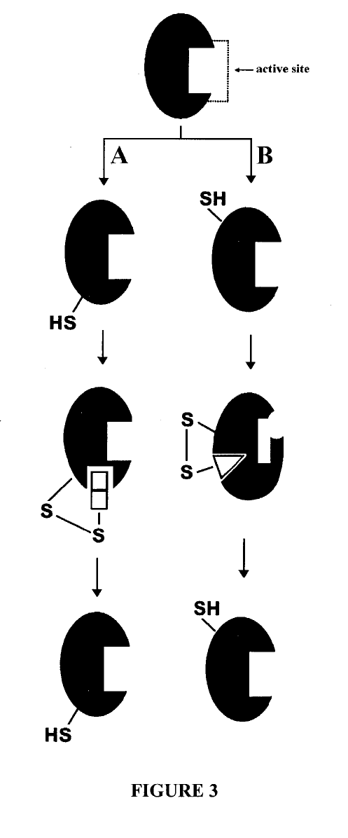

A schematic representation of the tethering method to identify exosites is shown in FIG. 3. In this embodiment, the primary binding site is depicted as an active site. FIG. 3A illustrates the situation where the exosite is an adaptive binding site. As can be seen, the exosite is induced by the presence of the ligand. However, the binding of a ligand to this exosite does not alter the conformation of the active site or alter function of the target. In contrast, FIG. 3B illustrates the situation where an exosite is identified that is also an allosteric site. As shown, the binding of a ligand to the allosteric exosite alters the conformation of the active site such that it inhibits the function of the target. In both cases, the target-compound conjugate optionally can be contacted with reducing agent to regenerate the target. In the case where the exosite is an allosteric exosite, the removal of the ligand reverses the change that occurred from the ligand binding to the allosteric exosite.

The method as shown in FIG. 3 is applied by making cysteine mutants of the desired target. A cysteine residue is introduced on a protein target at or near sites of interest. In one embodiment, sites of interest are chosen so that locations on the target are explored systematically. In another embodiment, sites of interest are near interface regions when the target is composed of multiple subunits. These subunits may be composed of the same polypeptide (e.g., homodimers) or different polypeptides (e.g. heterodimers). A cysteine is considered to be near the site of interest if it is located within 10 Angstroms from the site of interest, preferably within 5 Angstroms from the site of interest. If the target includes naturally occurring cysteine outside of the site of interest, they can optionally be mutated to another residue such as alanine to eliminate the possibility of dual labeling.

In general, residues to be mutated into a cysteine residue are solvent-accessible. Solvent accessibility may be calculated from structural models using standard numeric (Lee, B. & Richards, F. M. J. Mol. Biol 55:379-400 (1971); Shrake, A. & Rupley, J. A. J. Mol. Biol. 79:351-371 (1973)) or analytical (Connolly, M. L. Science 221:709-713 (1983); Richmond, T. J. J. Mol. Biol. 178:63-89 (1984)) methods. For example, a potential cysteine variant is considered solvent-accessible if the combined surface area of the carbon-beta (CB), or sulfur-gamma (SG) is greater than 20 Å² when calculated by the method of Lee and Richards (Lee, B. & Richards, F. M. J. Mol. Biol 55:379-400 (1971)). This value represents approximately 33% of the theoretical surface area accessible to a cysteine side-chain as described by Creamer et al. (Creamer, T. P. et al. Biochemistry 34:16245-16250 (1995)).

It is also preferred that the residue to be mutated to cysteine, or another thiol-containing amino acid residue, not participate in hydrogen-bonding with backbone atoms or, that at most, it interacts with the backbone through only one hydrogen bond. Wild-type residues where the side-chain participates in multiple (>1) hydrogen bonds with other side-chains are also less preferred. Variants for which all standard rotamers (chil angle of −60°, 60°, or 180°) can introduce unfavorable steric contacts with the N, CA, C, O, or CB atoms of any other residue are also less preferred. Unfavorable contacts are defined as interatomic distances that are less than 80% of the sum of the van der Waals radii of the participating atoms. In certain embodiments where the site of interest is a concave region, residues found at the edge of such a site (such as a ridge or an adjacent convex region) are more preferred for mutating into cysteine residues. Convexity and concavity can be calculated based on surface vectors (Duncan, B. S. & Olson, A. J. Biopolymers 33:219-229 (1993)) or by determining the accessibility of water probes placed along the molecular surface (Nicholls, A. et al. Proteins 11:281-296 (1991); Brady, G. P., Jr. & Stouten, P. F. J. Comput. Aided Mol. Des. 14:383-401 (2000)). Residues possessing a backbone conformation that is nominally forbidden for L-amino acids (Ramachandran, G. N. et al J. Mol. Biol. 7:95-99 (1963); Ramachandran, G. N. & Sasisekharahn, V. Adv. Prot. Chem. 23:283-437 (1968)) are less preferred targets for modification to a cysteine. Forbidden conformations commonly feature a positive value of the phi angle.

Other preferred variants are those which, when mutated to cysteine and tethered as to comprise -Cys-SSR¹, would possess a conformation that directs the atoms of R¹ towards the site of interest. Two general procedures can be used to identify these preferred variants. In the first procedure, a search is made of unique structures (Hobohm, U. et al. Protein Science 1:409-417 (1992)) in the Protein Databank (Berman, H. M. et al. Nucleic Acids Research 28:235-242 (2000)) to identify structural fragments containing a disulfide-bonded cysteine at position j in which the backbone atoms of residues j−1, j, and j+1 of the fragment can be superimposed on the backbone atoms of residues i−1, i, and i+1 of the target molecule with an RMSD of less than 0.75 squared Angstroms. If fragments are identified that place the C β atom of the residue disulfide-bonded to the cysteine at position j closer to any atom of the site of interest than the C β atom of residue i (when mutated to cysteine), position i is considered preferred. In an alternative procedure, the residue at position i is computationally “mutated” to a cysteine and capped with an S-Methyl group via a disulfide bond.

Various recombinant, chemical, synthesis and/or other techniques can be employed to modify a target such that it possesses a desired number of free thiol groups that are available for tethering. Such techniques include, for example, site-directed mutagenesis of the nucleic acid sequence encoding the target polypeptide such that it encodes a polypeptide with a different number of cysteine residues. Particularly preferred is site-directed mutagenesis using polymerase chain reaction (PCR) amplification (see, for example, U.S. Pat. No. 4,683,195 issued Jul. 28, 1987; and Current Protocols In Molecular Biology, Chapter 15 (Ausubel et al., ed., 1991)). Other site-directed mutagenesis techniques are also well known in the art and are described, for example, in the following publications: Ausubel et al., supra, Chapter 8; Molecular Cloning: A Laboratory Manual., 2nd edition (Sambrook et al., 1989); Zoller et al., Methods Enzymol. 100:468-500 (1983); Zoller & Smith, DNA 3:479-488 (1984); Zoller el al., Nucl. Acids Res., 10:6487 (1987); Brake et al., Proc. Natl. Acad. Sci. USA 81:4642-4646 (1984); Botstein et al., Science 229:1193 (1985); Kunkel et al., Methods Enzymol. 154:367-82 (1987), Adelman et al., DNA 2:183 (1983); and Carter et al., Nucl. Acids Res., 13:4331 (1986). Cassette mutagenesis (Wells et al., Gene, 34:315 [1985]), and restriction selection mutagenesis (Wells et al., Philos. Trans. R. Soc. London SerA, 317:415 [1986]) may also be used.

Amino acid sequence variants with more than one amino acid substitution may be generated in one of several ways. If the amino acids are located close together in the polypeptide chain, they may be mutated simultaneously, using one oligonucleotide that codes for all of the desired amino acid substitutions. If, however, the amino acids are located some distance from one another (e.g. separated by more than ten amino acids), it is more difficult to generate a single oligonucleotide that encodes all of the desired changes. Instead, one of two alternative methods may be employed. In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant.

In another aspect of the present invention, methods for identifying allosteric inhibitors are provided comprising:

a) performing a first tethering experiment and

b) performing a second tethering experiment wherein both tethering experiments comprise:

i) providing a target comprising a first binding site, a second binding site, and a chemically reactive group at or near the second binding site;

ii) contacting the target with a compound that is capable of forming a covalent bond with the chemically reactive group;

iii) forming covalent bond between the target and the compound thereby forming a target-compound conjugate; and;

iv) identifying the target-compound conjugate wherein the first tethering experiment is performed in the presence of a ligand that binds to the first binding site and the second tethering experiment is performed in the absence of the ligand that binds to the first binding site.

Compounds that form target-compound conjugate in the absence of the substrate or ligand but not in the presence of the substrate or ligand are candidates for allosteric inhibitors. In one embodiment, the target is an enzyme and the ligand that binds to the first binding site is a known competitive inhibitor. In another embodiment, the covalent bond is a disulfide and the compound is a ligand candidate possessing a masked disulfide.

In another aspect of the present invention, methods are provided for identifying allosteric inhibitors in a target capable of allosteric regulation. The method relies on disabling the allosteric site so that the binding of a ligand to the allosteric site no longer inhibits the target. The method comprises:

a) providing a target that is capable of allosteric regulation and a mutant thereof that is not capable of allosteric regulation;

b) contacting the target with a compound;

c) contacting the mutant with the compound; and

d) comparing the activity of the compound against the target with the activity of the compound against mutant.

In one embodiment, the allosterically disabled mutant possesses a mutation in; at least one residue that comprises the allosteric site. In another embodiment, the allosterically disabled mutant possesses mutations in at least two residues that comprise the allosteric site. In yet another embodiment, the allosterically disabled mutant possesses mutations in at least three residues that comprise the allosteric site.

Caspases

Caspases (cysteineyl aspartate-specific proteases) are a family of intracellular cysteine proteases that play pivital roles in both cytokine maturation and apoptosis. Like many other proteases, caspases are synthesized as inactive zymogens. These zymogens contain an N-terminal prodomain and a cleavage site that when cleaved results in a large subunit and a small subunit domains. Generally, the initial cleavage of an Asp-X bond separates the short C-terminal small subunit that allows the assembly of an active protease and cleavage of its own prodomain. The active form of these enzymes is a heterotetramer comprised of two large subunits and two small subunits. However, because the large and small subunits derive from the same polypeptide, the active form is often referred to (as it will herein) as a homodimer.

Using the methods herein, a novel allosteric site has been identified in the dimer interface of caspases. This site was first identified in caspase-3 and was believed to exist in other caspases due to the remarkable structural similarity within the caspase family of enzymes. For example, despite the relatively low sequence identity between caspase-3 to caspase-1 (29% identity) and caspase-9 (24% identity), the three enzymes share a high degree of structural similarity and are essentially superimposable with each other. See Mittl et al., J Biol Chem 272: 6539; Rotonda et al., Nat Struct Biol 3: 619; Chai et al., Proc. Natl. Acad. Sci (USA), 98:14250-14255; and Watt et al, Structure 7: 1135-1143. As it will be further described, the caspase allosteric site has been identified in other caspases. However, because the caspase allosteric site was first characterized in caspase-3, the residues that comprise the caspase allosteric site are described using the caspase-3 numbering scheme.

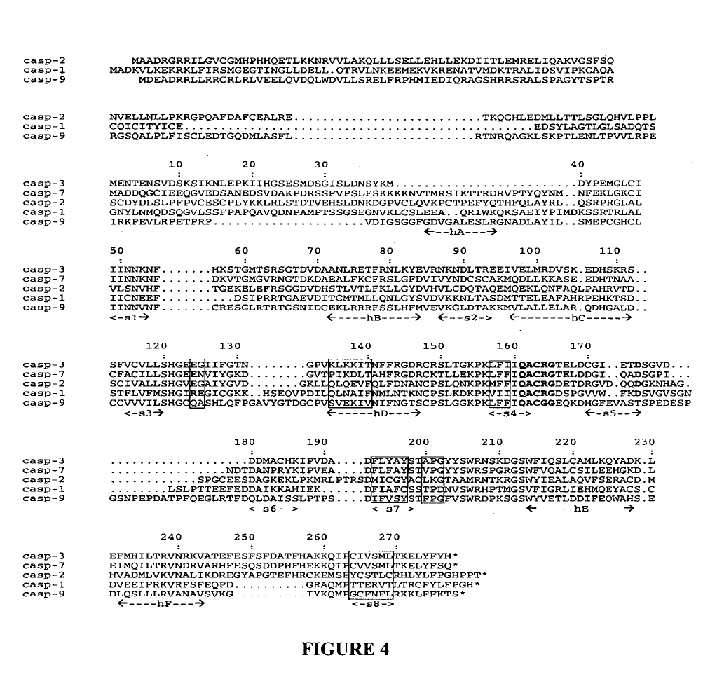

A sequence alignment of caspase-3 with selected representative caspases is shown in FIG. 4. This alignment was generated using the following amino acid sequences for the indicated caspases: XP_{—}054686 (caspase-3); NP_{—}150634 (caspase-1); NP_{—}116764 (caspase-2); AAH15799 (caspase-7); and AAH02452 (caspase-9). Aligning residues between the caspase-3 sequence and caspases-1, -2, -7, and -9 sequences respectively are said to correspond to each other. For example Cys-264 is the 264th amino acid residue in caspase-3 and corresponds to a threonine in caspase-1, to a tyrosine in caspase-2, a cysteine in caspase-7, and a glycine in caspase-9.

Other caspases can be aligned with reference to the alignment shown in FIG. 4. Alternatively, the sequences can be aligned with standard alignment software such as Clustal W (1.81) (http://www2.ebi.ac.uk/clustalw/).

In one embodiment, the caspase allosteric site comprises residues in a caspase that are within 5 Angstroms of a residue corresponding to Cys-264 in caspase-3. A residue is said to be within 5 Angstroms if any of its atoms is 5 Angstroms or less from any atom of the residue corresponding to Cys-264 in caspase-3. In another embodiment, the caspase allosteric site comprises residues in a caspase that are within 3 Angstroms or less from any atom of the residue corresponding to Cys-264 in caspase-3.

In another embodiment, the caspase allosteric site comprises at least two residues corresponding to the residues of caspase-3: Glu-124; Gly-125; Lys-135; Leu-136; Lys-137, Lys-138; Ile-139; Thr-140; Leu-157; Phe-158; Ile-159; Phe-193, Leu-194; Tyr-195; Ala-196; Tyr-197; Ala-200; Pro-201; Gly-202; Cys-264; Ile-265; Val-266; Ser-267; Met-268; and Leu-269. These caspase-3 residues and corresponding residues in caspase-1, caspase-2 caspase-7 and caspase-9 are boxed in FIG. 4. In another embodiment, the caspase allosteric site comprise at least two residues corresponding to the residues of caspase-3Cys-264; Ile-265; Val-266; Ser-267; Met-268; and Leu-269.

Caspase-3

Caspase-3 was cloned and mutants Where cysteine residues are introduced at various locations throughout the protein were made. Example 1 describes the cloning and mutatagenesis for an illustrative set of cysteine mutants in greater detail. The cloned and mutant proteins were characterized using a tetrapeptide enzymatic assay as described in Example 2.

Tethering experiments were performed using caspase-3 and cysteine mutants thereof as described in Example 3 with a library of ligand candidates of the formula

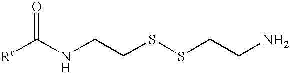

where R^{c} is as previously defined. During the course of these tethering experiments, an allosteric site on caspase-3 was discovered in the vicinity of naturally occurring cysteine in the small subunit, Cys-264.

The identification of Cys264 as the naturally occurring cysteine being modified by the selected ligand candidates is described in Example 4.

Two selected ligand candidates were compounds 1 and 2

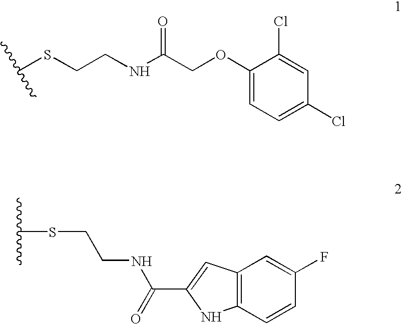

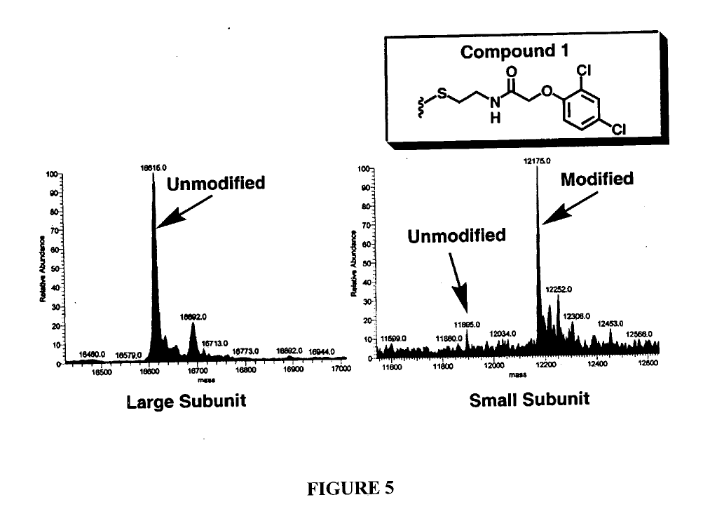

The portion depicted correspond to the R^{c}C(═O)NHCH₂CH₂S— portion of the ligand candidates that forms the disulfide bond with Cys264. FIG. 5 is a representative tethering experiment showing compound 1 forming a disulfide bond with the small subunit but not the large subunit of caspase-3.

Compounds 1 and 2 were strongly selected indicating that these compounds possessed an inherent binding affinity to the allosteric site. In addition, structure-activity relationships were observed from tethering experiments. For example, while compound 1 is strongly selected, compounds 3 and 4 are not.

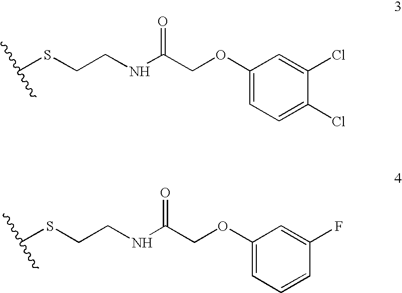

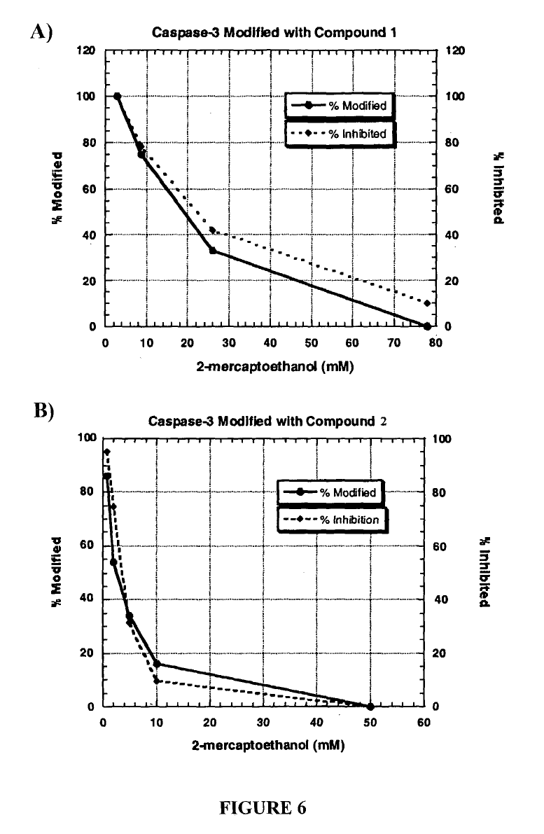

Using the assay described in Example 5, compounds 1 and 2 were further characterized and shown to inhibit caspase-3 activity in a stoichiometric manner. As can be seen in FIG. 6, the percent inhibition tracks with the percent of caspase-3 that forms a disulfide bond with compounds 1 or 2. Notably, this inhibition is reversible as demonstrated with the restoration of enzymatic activity upon the reduction of the disulfide bond between caspase-3 and compound 2.

The possibility that these compounds disrupt the formation of the active homodimer was investigated and eliminated. The elution profile of a sizing chromatograph following the conversion of the active homodimer to a monomer was essentially identical for caspase-3 in both the absence and presence of compound 1 or 2.

Instead, as demonstrated by structural experiments, the mechanism of action is due to a rearrangement in the active site upon binding of compound 1 or 2 to the allosteric site. Notably, the binding of compounds 1 or 2 to the allosteric exosite precludes substrate binding in the active site. Interestingly, the converse is also true. The binding of substrate to the active site precludes the binding of compounds 1 or 2 to the allosteric exosite. In other words, binding events to the active site and allosteric site are mutually exclusive.

Caspase-7

Based on a 53% sequence identity with caspase-3 and a cysteine located at a corresponding site as Cys-264, it was expected that the allosteric site in caspase-7 would behave similarly to that in caspase-3. After confirming that compounds 1 and 2 do inhibit caspase-7 in a similar manner to that in caspase-3, caspase-7 was selected for structural studies of the caspase allosteric site as it is the only caspase that has been crystallized in the pro-, active apo, and active-inhibited forms.

Example 6 describes the cloning and crystallization procedures for the structural studies of caspase-7 complexed with compound 1 and with compound 2. These compounds bind to a deep pocket in the dimer interface. Because this pocket is discernable even in the absence of compounds 1 or 2, the caspase allosteric site is not induced by the presence of an allosteric inhibitor. The structural studies of caspase-7 with compounds 1 or 2 confirm that these compounds bind specifically to the allosteric site and reveal a potential mechanism behind the allosteric inhibition.

Because the active form of caspases is a homodimer, two molecules of an allosteric inhibitor bind to the active complex, one molecule per each small subunit. The allosteric binding site formed by each of the two small subunits is spatially adjacent to each other.

The two molecules of compound 1 bind to their respective site and face each other in an anti-parallel orientation. Because the two nearest atoms between the two molecules are the respective carbonyl and are separated by a distance of 7 Angstroms, they do not appear to interact with each other. In fact, no direct hydrogen bond interaction was evident between the two molecules of compound 1 or between either molecule of compound 1 and the protein. However, five potential water mediate hydrogen bonds were identified between the two molecules of compound 1 and the protein.

In contrast, the two molecules of compound 2 interact with each other in an edge-to-edge fashion forming one intramolecular hydrogen bond between the indole nitrogen of one molecule and the carbonyl oxygen of the other. In addition, three other direct hydrogen bonds appear to be made between compound 2 and the protein.

The two different binding modes appear to correlate with the different ways that compound 1 and compound 2 exert their respective effect on the active site. In the case of compound 1, the compound binds to the allosteric site formed by the same polypeptide as the active site it inhibits. In the case of compound 2, the compound binds to the allosteric site formed by one polypeptide and inhibits the active site formed by the other polypeptide.

Nevertheless, the mechanism by which the binding of compound 1 or compound 2 effectuates inhibition at the active site appears to be the same. Activation of caspases requires cleavage of both a pro-peptide and cleavage between the large and small subunits. Although these cleavages render an “active” form of the protein, the resulting caspase is not catalytically competent until a structural rearrangement of the peptide-binding groove occurs.

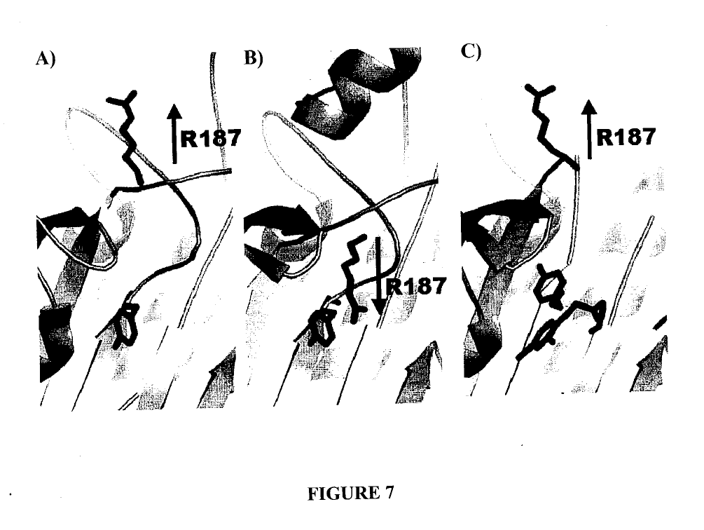

In the absence of a bound substrate, the so-called “active” form of the caspase remains in a catalytically inactive conformation. As shown in FIG. 7A, Arg-164 (using caspase-3 numbering), a residue immediately adjacent to the active site cysteine protrudes up into the active site such that the peptide-binding groove is not in a suitable conformation to bind substrate. When substrate is bound, a number of structural changes are induced. Arg-164 is forced down into the core of the protein and the peptide binding groove molds to fit the substrate (see FIG. 7B), both of which are required in the catalytically competent form of the enzyme.

In the structures of caspase-7 bound to either compound 1 or 2, Tyr-197 (using caspase-3 numbering) is displaced such that it precludes burial of Arg-164 in the core of the protein. As a result, Arg-164 protrudes up (see FIG. 7C) as seen in the unbound structure. In addition, the peptide-binding groove is disordered.

Caspase-9

Caspase-9 was also investigated for evidence of an allosteric site. Unlike caspase-7, caspase-9 shares only a 24% sequence identity with caspase-3. In addition, the residue that corresponds to Cys-264 in caspase-3 is a glycine and not a cysteine. However, a naturally occurring cysteine also occurs in the allosteric pocket but corresponds to Ile-265 in caspase-3.

Example 7 describes the procedures used for cloning and assaying caspase-9. As with caspase-3, tethering experiments identified several ligand candidates including the following

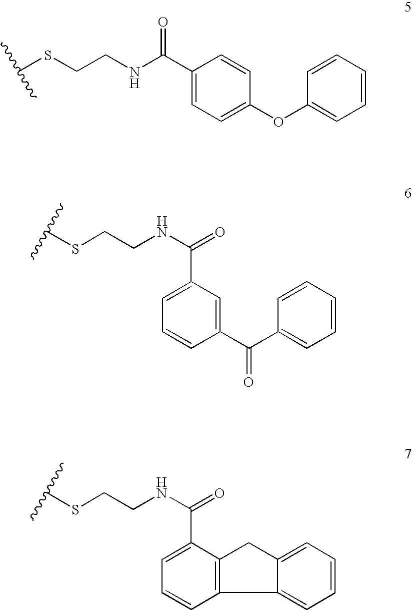

as binding to the small subunit. Because caspase-9 includes only one naturally occurring cysteine in the small subunit, it was readily identified as the one corresponding to corresponds to Ile-265 in caspase-3. Notably, this residue is almost in an identical location to Cys-264 in caspase-3.

As in the caspase-3 case, among the evidence that these compounds are specifically binding to the allosteric site was a discernable structure-activity relationship. For example, a ligand candidate of compound 8, having the structure

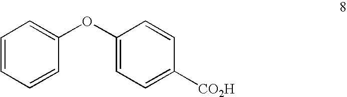

was also selected. This compound differs, in part, from compound 5 by the presence of an additional carboxyl group. However, other ligand candidates were tested and were found to be not selected. Certain functional groups substituted on the biaryl ether tested include compounds having an additional hydroxyl group, nitro group, etc . . . , or wherein the carboxyl group is substituted at different positions such as those shown below:

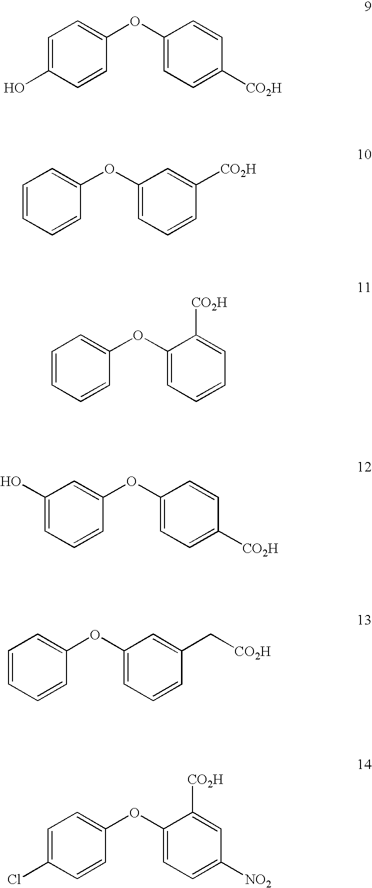

PTP-1B

In addition to the caspases, another previously unknown allosteric site has been identified in PTP-1B, a phosphatase that has become a highly validated target for the treatment of various metabolic disorders such as diabetes and obesity in recent years.

Human PTP-1B is a 435 amino acid protein. However, because the full-length protein does not express well in bacteria, studies of PTP-1B typically are carried out using truncated forms such as those corresponding to the first 321 or the first 298 amino acids of the protein. Example 8 describes protocols for making the truncated versions of PTP-1B and mutants thereof.

Crystallographic studies of PTP-1B in the presence and absence of various allosteric inhibitors have shown that unlike the allosteric site identified in caspases, the allosteric site in PTP-1B is adaptable. In other words, the allosteric binding site is not discernable in the absence of a suitable ligand.

An illustrative example of an allosteric inhibitor of PTP-1B is compound 15 which is shown below

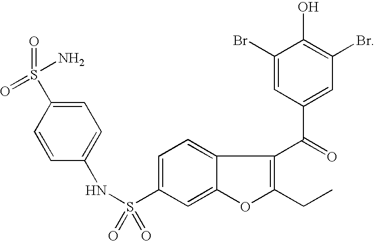

This compound binds to and inhibits PTP-1B non-competitively with an IC₅₀ of about 30 μM. An illustrative protocol for determining the activity of PTP-1B is described in Example 9. In the presence of an allosteric inhibitor like compound 15, a crevice is formed that is created by the following residues: Glu-186; Ser-187; Pro-188; Ala-189; Leu-192; Asn-193; Phe-196; Lys-197; Glu-200; Leu-272; Glu-276; Gly-277; Lys-279; Phe-280; Ile-281; and Met-282. These residues form a contiguous surface in which the compound binds. However, in the absence of an allosteric inhibitor, most of the site is occluded by the presence of a helix formed by residues 283-298 and the majority of the above residues are no longer accessible.

The large conformational change that occurs in the presence of an allosteric inhibitor is mediated by the interactions of at least three residues: Tyr-152, Asn-193, and Tryp-291, and is believed to be part of a regulatory mechanism for PTP-1B. In the absence of an allosteric inhibitor, the N62 of Asn-193 (one of the allosteric site forming residues) makes a hydrogen bond with the On of Tyr-152 and the helix formed by residues 283-298 is maintained in position at least in part from the non-bonded interactions of the indole ring of Trp-291 with the phenyl rings of Phe-280 and Phe-196 (both of which are also allosteric site forming residues). A fourth residue, Lys-197 (another allosteric site forming residue) is also believed to participate in maintaining the hydrogen bond interaction between the N_{δ2} of Asn-193 the Oη of Tyr-152.

In the presence of an allosteric inhibitor such as compound 15, the helix formed by residues 283-298 is displaced and/or disordered. In the case of compound 15, the benzofuran moiety displaces the indole ring of Trp-291. The carbonyl oxygen of compound 15 makes a hydrogen bond with N₆₂ of Asn-193 such that the N₆₂ of Asn-193 is no longer available for hydrogen bonding to Oil of Tyr-152. The disruption of the hydrogen bond between Asn-193 and Tyr-152 in part mediates a conformation change in the phenolic ring of Tyr-152. The rotation of the phenolic ring of Tyr-152 propagates a conformational change in the active site of PTP-1B that functionally inactivates the enzyme.

The importance of allosteric forming residues, Asn-193, Phe-196, and Phe-280 has been confirmed in part using mutagenesis experiments. When a mutant PTP-1B is made where Asn-193 is mutated to alanine, Phe-196 is mutated to arginine, and Phe-280 is mutated to cysteine, the allosteric mechanism is disabled and allosteric inhibitors such as compound 15 is no longer capable of inhibiting the enzyme.

The invention is further illustrated by the following non-limiting examples.

### EXAMPLE 1

Cloning of Human Caspase-3

The human version of caspase-3 (also known as Yama, CPP32 beta) was cloned directly from Jurkat cells (Clone E6-1; ATCC). Briefly, total RNA was purified from Jurkat cells growing-at 37° C./5%CO₂ using Tri-Reagent (Sigma). Oligonucleotide primers were designed to allow DNA encoding the large and small subunits of Caspase-3/Yama/CPP32 to be amplified by polymerase chain reaction (PCR). Briefly, DNA encoding amino acids 28-175 (encompassing most of the large subunit) was directly amplified from 1 μg total RNA using Ready-To-Go-PCR Beads (Amersham/Pharmacia) and the following oligonucleotides:

(SEQ ID NO: 1)

5′-TTCCATATGTCTGGAATATCCCTGGACAACAGTTA-3′
and

(SEQ ID NO: 2)

5′-AAGGAATTCTTAGTCTGTCTCAATGCCACAGTCCAG-3′.

DNA encoding amino acids 176-277 (encompassing most of the small subunit) was directly amplified from 1 μg total RNA using Ready-To-Go-PCR Beads (Amersham/Pharmacia) and the following oligonucleotides:

(SEQ ID NO: 3)

5′-TTCCATATGAGTGGTGTTGATGATGACATGGCG-3′
and

(SEQ ID NO: 4)

5′-AAGGAATTCTTAGTGATAAAAATAGAGTTCTTTTGTGAG-3′

Amplified DNA corresponding to either the large subunit or the small subunit of caspase-3 was then cleaved with the restriction enzymes EcoRI and NdeI and directly cloned using standard molecular biology techniques into pRSET-b (Invitrogen) digested with EcoRI and NdeI. [See e.g. Tewari M, Quan LT, O'Rourke K, Desnoyers S, Zeng Z, Beidler D R, Poirier G G, Salvesen G S and Dixit V M. Yama/CPP32 beta, a mammalian homolog of CED-3, is a CrmA-inhibitable protease that cleaves the death substrate poly (ADP-ribose) polymerase Cell 81. (5), 801-809 (1995)].

There are the two reported protein sequences for the small subunit, and each differ by a single amino acid, having either an Aspartic acid (GenBank accession #P42574) or a Glutamic acid (GeneBank accession #XP_{—}054686) at amino acid position 190 (relative to the active site Cysteine being position 163). Both forms were successfully cloned, expressed and purified, and were functionally indistinguishable.

Preparation of Single Stranded DNA

Plasmids containing DNA encoding either the large or small subunits of Caspase-3 were separately transformed into E. coli K12 CJ236 cells (New England BioLabs) and cells containing each construct were selected by their ability to grow on ampicillin containing agar plates. Overnight cultures of the large and small subunits were individually grown in 2YT (containing 100 μg/mL of ampicillan) at 37° C. Each culture was diluted 1:100 and grown to A₆₀₀=0.3-0.6. A 1.5 mL sample of each culture was removed and infected with 10 μL of phage VCS-M13 (Stratagene), shaken at 37° C. for 60 minutes, and an overnight culture of each was prepared with 1 mL of the infected culture diluted 1:100 in 2YT with 100 μg/mL of ampicillan and 20 μg/ml of chloramphenicol and grown at 37° C. Cells were centrifuged at 3000 rcf for 10 minutes and 1/5 volume of 20% PEG/2.5M NaCl was added to the supernatant. Samples were incubated at room temperature for 10 minutes and then centrifuged at 4000 rcf for 15 minutes. The phage pellet was resuspended in PBS and spun at 15 K rpm for 10 minutes to remove remaining particulate matter. Supernatant was retained, and single stranded DNA was purified from the supernatant following procedures for the QIA prep spin M13 kit (Qiagen).

Identification of Residues to be Modified to Cysteine Residues

Selection of amino acid residues that were modified to cysteine residues was made by examining the three-dimensional crystal structure of caspase-3. Nine different amino acid residues were chosen for modification to cysteine residues. Each version of caspase-3 harboring cysteine mutations was expressed at high levels in E. coli cells (generally >1 mg/l). In all but one case we were able to successfully purify correctly refolded tetrameric protein (as assessed by its ability to be purified by Uno-5 Q chromatography). However, caspase-3 protein containing a histidine to cysteine mutation at amino acid 121 of the large subunit could not be purified by conventional chromatography. Since we were able to purify each subunit individually, we reasoned that this was most likely due to the inability of this variant form of caspase-3 to correctly form a tetramer (i.e. to refold the large with the small subunit). We also found that not all versions of caspase-3 bearing novel cysteine residues were catalytically active, for instance, Y204C is catalytically inactive.

Single Stranded Mutagenesis

Illustrative examples of cysteine mutants within the small subunit include F256C; S209C; S251C; W214C; and Y204C. These mutants were made with the following primers:

| | |
|---|---|
| F256C | |
| (5′-CTT TGC ATG ACA AGT AGC GTC-3′), | (SEQ ID NO: 5) |
| S209C | |
| (5′-GCC ATC CTT ACA ATT TCG CCA-3′), | (SEQ ID NO: 6) |
| S251C | |
| (5′-AGC GTC AAA GCA AAA GGA CTC-3′), | (SEQ ID NO: 7) |
| W214C | |
| (5′-CTG GAT GAA ACA GGA GCC ATC-3′), | (SEQ ID NO: 8) |
| and | |
| Y204C | |
| (5′-TCG CCA AGA ACA ATA ACC AGG-3′). | (SEQ ID NO: 9) |

Illustrative examples of cysteine mutants within the large subunit include H121C; L168C; M61C; and S65C. These mutants were made with the following primers:

(SEQ ID NO: 10)

| | |
|---|---|
| H121C | (5′-TTC TTC ACC ACA GCT GAG AAG-3′), |

(SEQ ID NO: 11)

| | |
|---|---|
| L168C | (5′-GCC ACA GTC ACA TTC TGT ACC-3′), |

(SEQ ID NO: 12)

| | |
|---|---|
| M61C | (5′-CCG AGA TGT ACA TCC AGT GCT-3′), |
| and | |

(SEQ ID NO: 13)

| | |
|---|---|
| S65C | (5′-ATC TGT ACC ACA CCG AGA TGT-3′). |

Approximately 100 pmol of each primer was phosphorylated by incubating at 37° C. for 60 minutes in buffer containing 1×TM Buffer (0.5M Tris pH 7.5, 0.1M MgCl₂), 1 mM ATP, 5 mM DTT, and 5U T4 Kinase (NEB). Kinased primers were annealed to the template DNA in a 20 μL reaction volume (˜50 ng kinased primer, 1×TM Buffer, and 10-50 ng single-stranded DNA) by incubation at 85° C. for 2 minutes, 50° C. for 5 minutes, and then at 4° C. for 30-60 minutes. An extension cocktail (2 mM ATP, 5 mM dNTP's, 30 mM DTT, T4 DNA Ligase (NEB), and T7 Polymerase (NEB)) was added to each annealing reaction and incubated at room temperature for 3 hours. Mutagenized DNA was transformed into E. coli XLI-Blue cells, and colonies containing plasmid DNA selected were for by growth on LB agar plates containing 100 μg/ml ampicillin. DNA sequencing was used to identify plasmids containing the appropriate mutation.

Protein Expression and Purification

Plasmid DNA encoding cysteine mutations in the large subunit were transformed into Codon Plus BL21 Cells and plasmid DNA encoding cysteine mutations in the small subunit were transformed into BL21 (DE3) pLysS Cells. Codon Plus BL21 Cells containing plasmids encoding wild-type and cysteine mutated versions of the large subunit were grown in 2YT containing 150 μg/mL of ampicillan overnight at 37° C. and immediately harvested. BL21 pLysS Cells containing plasmids encoding wild-type and cysteine mutated versions of the small subunit were grown in 2YT at 37° C. with 150 μg/mL of ampicillan until A₆₀₀=0.6. Cultures were subsequently induced with 1 mM IPTG and grown for an additional 3-4 hours at 37° C. After harvesting cells by centrifuging at 4K rpm for 10 minutes, the cell pellet was resuspended in Tris-HCl (pH 8.0)/5 mM EDTA and micro fluidized twice. Inclusion bodies were isolated by centrifugation at 9K rpm for 10 minutes and then resuspended in 6M-guanidine hydrochloride. Denatured subunits were rapidly and evenly diluted to 1001 g/mL in renaturation buffer (100 mM Tris/KOH (pH 8.0), 10% sucrose, 0.1% CHAPS, 0.15M NaCl, and 10 mM DTT) and allowed to renature by incubation at room temperature for 60 minutes with slow stirring.

Renatured proteins were dialyzed overnight in buffer containing 10 mM Tris (pH 8.5), 10 mM DTT, and 0.1 mM EDTA. Precipitate was removed by centrifuging at 9K rpm for 15 minutes and filtering the supernatent through a 0.22 μm Cellulose Nitrate filter. The supernatant was then loaded onto an anion-exchange column (Uno5 Q-Column (BioRad)), and correctly folded caspase-3 protein was eluted with a 0-0.25 M NaCl gradient at 3 mL/minute. Aliquots of each fraction were electrophoresised on a denaturing polyacrylamide gel and fractions containing Caspase-3 protein were pooled.

### Example 2

This example describes one method for characterizing the enzymatic activity of caspase-3.

A coumarin-based fluorogenic substrate that incorporated the optimal tetrapeptide recognition motif for caspase-3 was purchased from Alexis Biochemicals. Caspase-3 was added to 1× reaction buffer (25 mM HEPES pH 7.4, 0.1% CHAPS, 50 mM KCl and 5 mM P-Mercaptoethanol) to a final concentration of 1.6 nM. The tetrapeptide substrate (Ac-Asp-Glu-Val-Asp-AFC) was added to a final concentration of 5 μM bringing the final reaction volume to 50 μL. Assays were carried out in black 96-well flat bottom, polystyrene plates (Corning) and caspase activity was monitored using Molecular Devices' Microplate Spectrofluorometer Gemini XS with an excitation wavelength of 365 nm and an emission wavelength of 495 nm. Kinetic data was collected over a 15-minute assay run at room temperature.

### Example 3

This example describes one embodiment of a tethering experiment using caspase-3 or cysteine mutants thereof. Tethering screens were typically carried out in a 50 μl volume with a final concentration of 1-5 μM caspase-3,1-20 mM β-Mercaptoethanol, and 1-2 mM ligand candidates (total concentration of all ligand candidates in the pool; thus each ligand candidate in a pool of ˜10 had a final concentration of ˜100-200 μM) in TE buffer (10 mM Tris, 1 mM EDTA, pH 8.0). Reactions were allowed to proceed to equilibrium (≧1 hour) before being analyzed by mass spectrometry. The reaction mixture was loaded onto a Finnegan LCQ2 or LCQ3 LCMS, with each run taking 2-5 minutes, depending upon the separation procedure. After deconvolution, the large and/or small subunits were identified based upon their known molecular weight.

### Example 4

This example describes the identification of Cys264 as the naturally occurring cysteine to which compounds 1 and 2 form a disulfide bond with caspase-3. The small subunit of caspase 3 includes three cysteines (Cys184, Cys220, and Cys264). Of these three, Cys220 is buried and thus was eliminated as a possibility.

In one experiment, mutants where made where either the cysteine at position 184 and 264 were mutated to serine using the appropriate DNA primers (C184S 5′-TAT TTT ATG AGA CGC CAT GTC-3′ (SEQ ID. NO. 14); C264S 5′-GGA AAC AAT CGA TGG AAT CTG-3′ (SEQ ID. NO 15), where the underlined triplet indicates the introduced serine residue). The clones subsequently were confirmed by DNA sequence analysis. The C184S mutant were able to form a target-compound conjugate with compounds 1 and 2 but the C264S mutant was not.

The identification of Cys-264 as the cysteine residue was further confirmed by peptide mapping of the target-compound conjugate with compound 1. Caspase-3 (4 AM) in TE buffer pH 8.0 was incubated in the presence of compound 1 (200 μM) and β-mercaptoethanol (1 mM) for one hour at 25° C. When 100% of the small subunit was modified with compound 1 (as determined by LC/MS), excess compound 1 and reductant were removed by size exclusion chromatography on a sephadex G-25 column. To ensure that trace amounts of reductant or unbound compound 1 were removed, the protein was diluted 10-fold in TE buffer and reconcentrated in a Millipore 5,000 MWCO filtration device. This process was repeated three times. Modified caspase-3 was heat denatured at 98° C. for 1 minute, then incubated on ice until the solution reached room temperature. Following heat treatment, approximately 70% of caspase-3 remained as a target-compound conjugate. The target-compound conjugate was digested by endoproteinase Glu-c (20 ng/μL) in 500 mM ammonium acetate buffer pH 4.0 for 20 hours at room temperature. Peptide masses were analyzed LC/MS on a Q-STAR apparatus. The masses of each of the predicted digestion fragments, including the peptide containing Cys264 covalently linked to compound 1 were observed. Peptides masses corresponding to Cys184 or Cys220 covalently linked to compound 1 were not observed. This was further confirmed by observation of the tripeptide P₂₆₃C₂₆₄I₂₆₅+compound 1 after fragmentation by MS/MS.

### Example 5

This example describes one embodiment for correlating the degree of disulfide formation (the formation of the target-ligand conjugate) with degree of inhibition of caspase-3 enzymatic activity.

Using a constant concentration of protein (1-5 μM) and compound (typically 200 μM), the concentration of β-mercaptoethanol in the reaction was varied to modulate the extent of the formation of the target-ligand conjugate. After approximately 1 hour, the samples were examined by LC/MS to determine the percentage of small subunit modified by compound 1 at each β-Mercaptoethanol concentration. At the same time, 1 μL of the sample was removed and added to 199 μl of 1× reaction buffer (25 mM HEPES pH 7.4, 0.1% CHAPS, 50 mM KCl and 5 mM β-Mercaptoethanol containing 5 μM Ac-Asp-Glu-Val-Asp-AFC). For analysis of compound 1, 200 μM compound and various concentrations of β-mercaptoethanol were also added, so that the final β-mercaptoethanol concentration remained the same as in the tethering reaction. After dilution, the final caspase-3 enzyme concentration was ˜5 nM. Caspase activity was monitored using a Molecular Devices' Microplate Spectrofluorometer Gemini XS with an * excitation wavelength of 365 nm and an emission wavelength of 495 nm. The relative activity of caspase-3 modified with various amounts of compound 1 was compared to the activity observed under similar reaction conditions but in the absence of compound 1. Compound 2 was similarly investigated.

### Example 6

This example describes the cloning and cystallization procedures for the structural studies of caspase-7 complexed with compound 1 and with compound 2.

As detailed in Table 1, a series of plasmids for expression of the large subunit of caspase-7 was created by sub-cloning the coding sequence for caspase-7 residues 50-198 or 57-198 into pRSET (amp^{r}, Invitrogen) or pET3a (amp^{r}, Novagen).

TABLE 1

| Plasmid | Vector | Insert |
|---|---|---|
| pJH02 | pRSET | casp-7 large (aa50 − 198) |
| pJH03 | pRSET | casp-7 large (aa57 − 198) |
| pJH05 | pET3a | casp-7 large (aa57 − 198) |
| pJH06 | pBB75 | casp-7 small (aa199 − 303 + QLHis6) |
| pJH07 | pBB75 | casp-7 small (aa210 − 303 + QLHis6) |
| pJH08 | pRSET | casp-7 large (aa57 − 198) D192A (parent JH03) |
| pJH09 | pRSET | casp-7 large (aa50 − 198) D192A (parent JH02) |
| pJH11 | pET3a | casp-7 large (aa57 − 198) D192A ( arent JH05) |

For expression of the small subunit, the coding sequence for caspase-7 residues 199-303 plus the amino acids QLHHHHHH or 210-303 with the same addition was ligated into pBB75 (kan^{r}) (Batchelor, Piper et al. 1998). The mutation D192A was also introduced into the large subunit to minimize heterogeneity. Plasmids pJH02, 03, 05, 08, 09 or 11 (Table 2) were transformed in combination with pJH06 or 07 and over-expression tests were performed. Of these combinations, pJH07 and 08 or pJH07 and 09 were the most highly over-expressed and readily purified using methods described by Chai et al., Cell 104: 769-80 (2001) and Chai et al., Cell 107: 399-407.

Caspase-7 (D192A) expressed from pJH07 and pJH08 (10 μM) was labeled by compound 1 (100 μM) by incubation at room temperature for one week in TE buffer (10 mM Tris 8.0, 1 mM EDTA) containing 500 μM β-mercaptoethanol. Labeling of the small subunit was 98% complete after several hours, as determined by mass spectroscopy, but was allowed to proceed longer to obtain 100% complete labeling. Labeling of caspase-7 (D192A) by compound 2 (50 μM) proceeded in the presence of 250 EM β-mercaptoethanol in TE buffer. Labeling was 60% complete after 4 hours, and 100% complete by 1 week. These proteins were transferred by buffer exchange in an NAP-5 column (Amersham Pharmacia Biotech AB) to a buffer containing 100 mM NaCl, 10 mM Tris pH 8.0 for crystallization. Labeled protein was concentrated to 12 mg/mL in a Millipore 5K MWCO concentration device.

Crystals of caspase-7 (D192A)/compound 1 formed in one week by hanging-drop vapor diffusion at 4° C. from a drop containing 1 μL protein and 2 μL of a mother liquor solution (100 mM citrate buffer pH 5.8, 1 M LiSO4, 1 M NaCl). Crystals of caspase-7/compound 2 grew from drops that were 1 μL protein and 1 μL mother liquor. Crystals of caspase-7 (D192A) with either compound were transferred to a drop of the growth mother-liquor containing 20% glycerol and incubated overnight at 4° C. The crystals were then flash frozen in liquid nitrogen.

Data for the complex with compound 1 was collected on a Rigaku generator with an Raxis-4 detector. Data was processed with D*trek. The data for the complex with compound 2 was collected at SSRL Beamline 9-1 on a Quantum-315 CCD camera (ADSC). Data was processed with CCP4-mosflm and scala as described by Project, C. C., Acta Crysta. D. 50: 760-763 (1994). The structures were solved by direct molecular replacement using the structure of active caspase-7 (1K86.pdb) and rigid body refinement in CCP4-amore. The structures were refined by iterative rounds of molecular rebuilding in O (Jones et al., Acta Crystallogr A 47: 110-119 (1991)) and energy minimization in CCP4-refmac. The final data statistics are shown in Table 2.

TABLE 2

| | caspase-7 (D192A)/ | caspase-7 (D192A)/ |
|---|---|---|
| | compound 1 | compound 2 |
| Space group | P3₂2₁ | P3₂2₁ |
| Unit cell dimensions | a = b = 90.7 | a = b = 90.2 |
| | c = 185.4 | c = 186.6 |
| | α = β = 90.0 | α = β = 90.0 |
| | γ = 120.0 | γ = 120.0 |
| Resolution (Å) | 20.0-3.0 | 10.0-3.0 |
| Total observations | 41311 | 41163 |
| Unique observations | 18324 | 18250 |
| Data coverage | 99.9 | 99.9 |
| Rsym (outer shell) | 8.0 (38.4) | 9.4 (36.6) |
| R_{working}/R_{free} | 25.9/29.6 | 25.3/29.8 |

The electron density maps for both complexes clearly revealed the orientation of the compounds 1 and 2 interacting with the core of the protein. The ordered nature of the compounds confirms that these tethering compounds are bound in a specific manner. The temperature factors for the compounds are as low or lower than the surrounding atoms from the protein itself, indicating that the inhibitor molecules are very well ordered, and are not bound in a random or spurious manner.

### Example 7

Human caspase-9 was cloned, expressed and purified according to published procedures (Garcia-Calvo, M, et. al. 1998. Inhibition of Human Caspases by Peptide-based and Macromolecular Inhibitors, JBC 273 (49):32608-32613; Thomberry, N A, et. al. A combinatorial Approach Defines Specificities of Members of the Caspase Family and Granzyme B, JBC 272 (29): 17907-17911) and then tested for the appropriate enzyme activity. Caspase-9 enzyme was added to 1× reaction buffer (100 mM MES pH 6.5, 10% Glucose, 0.1% CHAPS, 10 mM DTT, and 100 mM NaCl). Substrate addition (Ac-Leu-Glu-His-Asp-AFC) to a final concentration of 200 μM initiated the reaction, bringing the final reaction volume to 50 μL. Assays were carried out in black 96-well flat bottom, polystyrene plates (Corning). Caspase activity was monitored using a Molecular Devices' Microplate Spectrofluorometer Gemini XS with an excitation wavelength of 365 nm and an emission wavelength of 495 nm. Kinetic data was collected over a 15-minute assay run at room temperature.

### Example 8

This example describes one embodiment for making truncated versions of wildtype human PTP-1B. A cDNA encoding the first 321 amino acids of human PTP-1B was isolated from human fetal heart total RNA (Clontech). Oligonucleotide primers corresponding to nucleotides 91 to 114 (For) and complementary to nucleotides 1030 to 1053 (Rev) of the PTP-I B cDNA (Genbank M31724.1, Chemoff, 1990) were synthesized and used to generate a DNA using the polymerase chain reaction.

The primer Forward incorporates an NdeI restriction site at the first ATG codon and the primer Rev inserts a UAA stop codon followed by an EcoRI restriction site after nucleotide 1053. cDNAs were digested with restriction nucleases NdeI and EcoRI and cloned into pRSETc (Invitrogen) using standard molecular biology techniques. The identity of the isolated cDNA was verified by DNA sequence analysis.

A shorter cDNA, PTP-1B 298, encoding amino acid residues 1-298 was generated using oligonuclotide primers Forward and Rev2 and the clone described above as a template in a polymerase chain reaction.

Rev2: 5′-TGC CGG AAT TCC TTA GTC CTC GTG GGA AAG CTC C (SEQ ID NO: 16)

The 321 amino acid form of human-PTP-1B is as
follows as:

SEQ ID NO. 17

MEMEKEFEQIDKSGSWAAIYQDIRHEASDFPCRVAKLPKNKNRNRYRDVS
PFDHSRIKLHQEDNDYINASLIKMEEAQRSYILTQGPLPNTCGHFWEMVW
EQKSRGVVMLNRVMEKGSLKCAQYWPQKEEKEMIFEDTNLKLTLISEDIK
SYYTVRQLELENLTTQETREILHFHYTTWPDFGVPESPASFLNFLFKVRE
SGSLSPEHGPVVVHCSAGIGRSGTFCLADTCLLLMDKRKDPSSVDIKKVL
LEMRKFRMGLIQTADQLRFSYLAVIEGAKFIMGDSSVQDQWKELSHEDLE
PPPEHIPPPPRPPKRILEPH

Mutants were made as follows. PTP-1B 321 in pRSETc (Invitrogen) was used as a template and T7 and RSETrev primers were used as “outside” primers. Mutagenesis primers were:

PTP-1B[321; N193A; F196R]:

SEQ ID. NO. 18

Fwd primer: 5′-TTC TTG GCG TTT CTT CGC AAA GTC CGA

SEQ ID. NO. 19

Rev primer: 5′-GAC TTT GCG AAG AAA CGC CAA GAA TGA
PTP-1B[321; F280C]

SEQ ID. NO. 20

Fwd primer: 5′-GGT GCC AAA TGC ATC ATG GGG

SEQ ID. NO. 21

Rev primer: 5′-CCC CAT GAT GCA TTT GGC ACC

PTP-1B[321; N193A; F196R; F280C] was generated by joining an NdeI-PstI fragment from PTP-IB[321; N193A; F196R], corresponding to residues 1-215, with a PstI-EcoRI fragment from PTP-1B[321; F280C], corresponding to residues 216-321.

PTP-1B[298; N193A; F196R; F280C] was generated by PCR using PTP-1B[321; N193A; F196R; F280C] as a template. T7 vector primer was used as forward primer and truncation at residue 298 was generated using the primer 5′-TGC CGG AAT TCC TTA GTC CTC GTG CGA AAG CTC C (SEQ ID. NO. 22).

PTP-1B[298; C215S] was generated using Kunkel mutagenesis and PTP-1B[298] as a template. The mutagenesis primer was:

5′-GATGCCTGCACTGGAGTGCACCACAAC SEQ ID. NO. 23

### Example 9

This example describes one illustrative method for determining the IC₅₀ of the compounds of the present invention against PTP-1B. Substrate, pNPP (Sigma), was dissolved at 4 mM in 1×HN buffer (50 mM HEPES pH 7.0; 100 mM NaCl; 1 mM DTT) and 83 ul was mixed with 2 ul DMSO or 2 ul compound in DMSO. The reaction was started by addition of PTP-1B (750 μg in standard assay conditions) in 15 μl 1×HN buffer. The rate of product formation (OD405 nm minus OD655 nm, BioRad Benchmark or Molecular Devices Spectramax 190) was measured every 30 seconds for 15 minutes at 25 degrees C., and data were analyzed by linear regression. For endpoint assays, the reaction was stopped after 15 min. with 50 μl 3M NaOH and OD405nm-OD655 nm was measured. For IC₅₀ determination, rates normalized relative to uninhibited controls were plotted against compound concentration and fitted using a 4 parameter non-linear regression curve fit (y=[(A−D)/(I+{x/C}\hat{ }B)]+D, Spectramax Software package).

All references cited throughout the specification are expressly incorporated herein by reference. While the present invention has been described * with reference to the specific embodiments thereof, it should be understood by those skilled in the art that various changes maybe made and equivalents may be substituted without departing from the true spirit and scope of the invention. In addition, many modifications may be made to adapt a particular situation, material, composition of matter, process, and the like. All such modifications are within the scope of the claims appended hereto.

What is claimed is:

1. A method of identifying an allosteric site comprising:
a) providing a target comprising a first binding site, a second binding site, and a chemically reactive group at or near the second binding site;
b) contacting the target with a compound that is capable of forming a covalent bond with the chemically reactive group;
c) forming a covalent bond between the target and the compound thereby forming a target-compound conjugate;
d) determining whether the target-compound conjugate possesses a change in the primary binding site as compared with the target.

2. The method of claim 1 wherein the chemically reactive group is a thiol.

3. The method of claim 1 wherein the chemically reactive group is a masked thiol.

4. The method of claim 3 wherein the masked thiol is in the form of a disulfide.

5. The method of claim 2 or 3 wherein the covalent bond is a disulfide bond and the contacting step occurs in the presence of a reducing agent.

6. The method of claim 5 wherein the compound is a ligand candidate selected from the group consisting of: 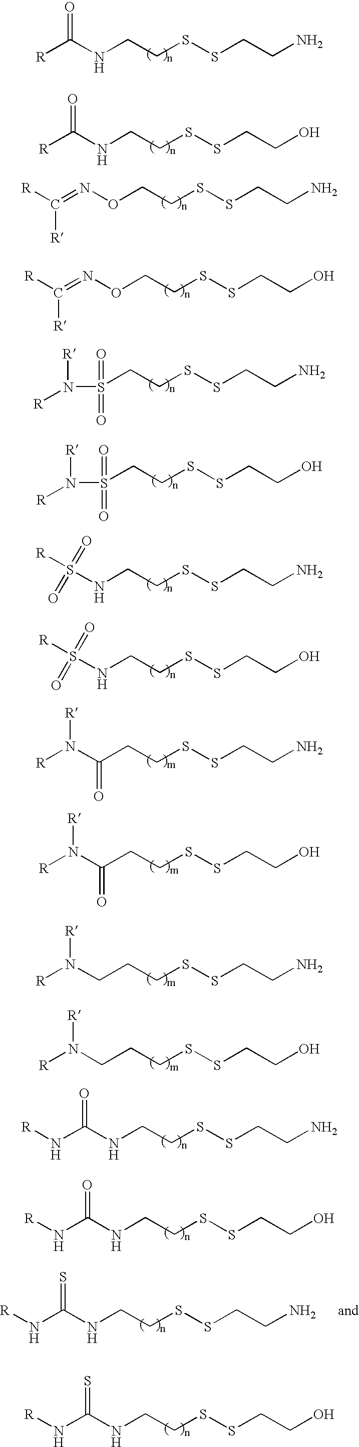
where R and R′ are each independently unsubstituted C₁-C₂₀ aliphatic, substituted C₁-C₂₀ aliphatic, unsubstituted aryl, or substituted aryl; m is 0, 1 or 2; and n is 1 or 2.

7. The method of claim 1 wherein the change is a functional change in the activity of the target.

8. The method of claim 1 wherein the change is a structural change.

9. The method of claim 8 wherein the structural change is determined using x-ray crystallography.

10. The method of claim 8 wherein the structural change is determined using NMR.

11. The method of claim 8 wherein the structural change is determined using circular dichroism.

12. The method of claim 1 wherein the target is a protease.

13. The method of claim 1 wherein the target is a kinase.

14. The method of claim 1 wherein the target is a phosphatase.

15. A method of identifying an allosteric inhibitor comprising:
a) performing a first tethering experiment and
b) performing a second tethering experiment wherein both tethering experiments comprise:
i) providing a target comprising a first binding site, a second binding site, and a chemically reactive group at or near the second binding site;
ii) contacting the target with a compound that is capable of forming a covalent bond with the chemically reactive group;
iii) forming covalent bond between the target and the compound thereby forming a target-compound conjugate; and
iv) identifying the target-compound conjugate wherein the first tethering experiment is performed in the presence of a ligand that binds to the first binding site and the second tethering experiment is performed in the absence of the ligand that binds to the first binding site.

16. The method of claim 15 wherein the chemically reactive group is a thiol or a masked thiol.

17. The method of claim 16 wherein the covalent bond is a disulfide bond and the contacting step occurs in the presence of a reducing agent.

18. The method of claim 17 wherein the compound is a ligand candidate selected from the group consisting of: 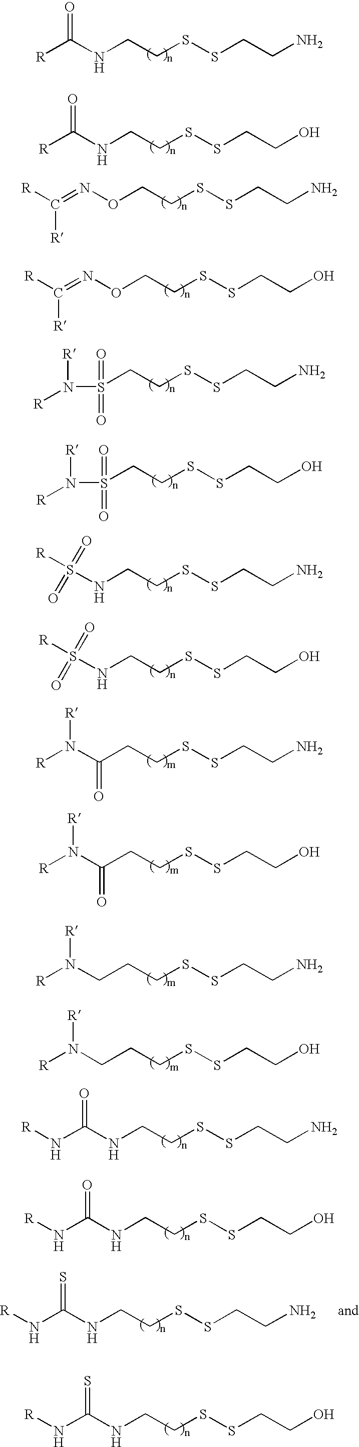
where R and R′ are each independently unsubstituted C₁-C₂₀ aliphatic, substituted C₁-C₂₀ aliphatic, unsubstituted aryl, or substituted aryl; m is 0, 1, or 2; and n is 1 or 2.

19. A method of identifying an allosteric inhibitor comprising:
a) providing a target that is capable of allosteric regulation and a mutant thereof that is not capable of allosteric regulation;
b) contacting the target with a compound;
c) contacting the mutant with the compound; and
d) comparing the activity of the compound against the target with the activity of the compound against mutant.

20. An allosteric inhibitor of a caspase.


SEQUENCE LISTING (23 sequences)

SEQ ID NO: 1 - DNA, 35 bp, homo sapiens
ttccatatgtctggaatatccctggacaacagtta

SEQ ID NO: 2 - DNA, 36 bp, homo sapiens
aaggaattcttagtctgtctcaatgccacagtccag

SEQ ID NO: 3 - DNA, 33 bp, homo sapiens
ttccatatgagtggtgttgatgatgacatggcg

SEQ ID NO: 4 - DNA, 39 bp, homo sapiens
aaggaattcttagtgataaaaatagagttcttttgtgag

SEQ ID NO: 5 - DNA, 21 bp, homo sapiens
ctttgcatgacaagtagcgtc

SEQ ID NO: 6 - DNA, 21 bp, homo sapiens
gccatccttacaatttcgcca

SEQ ID NO: 7 - DNA, 21 bp, homo sapiens
agcgtcaaagcaaaaggactc

SEQ ID NO: 8 - DNA, 21 bp, homo sapiens
ctggatgaaacaggagccatc

SEQ ID NO: 9 - DNA, 21 bp, homo sapiens
tcgccaagaacaataaccagg

SEQ ID NO: 10 - DNA, 21 bp, homo sapiens
ttcttcaccacagctcagaag

SEQ ID NO: 11 - DNA, 21 bp, homo sapiens
gccacagtcacattctgtacc

SEQ ID NO: 12 - DNA, 21 bp, homo sapiens
ccgagatgtacatccagtgct

SEQ ID NO: 13 - DNA, 21 bp, homo sapiens
atctgtaccacaccgagatgt

SEQ ID NO: 14 - DNA, 21 bp, homo sapiens
tattttatgagacgccatgtc

SEQ ID NO: 15 - DNA, 21 bp, homo sapiens
ggaaacaatcgatggaatctg

SEQ ID NO: 16 - DNA, 34 bp, homo sapiens
tgccggaattccttagtcctcgtgggaaagctcc

SEQ ID NO: 17 - protein, 320 aa, homo sapiens
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPH

SEQ ID NO: 18 - DNA, 27 bp, homo sapiens
ttcttggcgtttcttcgcaaagtccga

SEQ ID NO: 19 - DNA, 27 bp, homo sapiens
gactttgcgaagaaacgccaagaatga

SEQ ID NO: 20 - DNA, 21 bp, homo sapiens
ggtgccaaatgcatcatgggg

SEQ ID NO: 21 - DNA, 21 bp, homo sapiens
ccccatgatgcatttggcacc

SEQ ID NO: 22 - DNA, 34 bp, homo sapiens
tgccggaattccttagtcctcgtgcgaaagctcc

SEQ ID NO: 23 - DNA, 27 bp, homo sapiens
gatgcctgcactggagtgcaccacaac